(19) United States
(12) Patent Application Publication
(10) Pub. No.: US 2006/0024814 A1
(43) Pub. Date: Feb. 2, 2006

(54) Aptamer-functionalized electrochemical sensors and methods of fabricating and using the same

(76) Inventors: Kevin F. Peters, Corvallis, OR (US); Vincent T. Remcho, Corvallis, CO (US)

Correspondence Address: HEWLETT-PACKARD DEVELOPMENT COMPANY;Intellectual Property Administration, P.O. Box 272400, Fort Collins, CO, 80527-2400, US

(21) Appl. No.: 10/901,512

(22) Filed: Jul. 29, 2004

(51) Int. Cl. C12M 1/34

(52) U.S. Cl. 435/287.2

(57) ABSTRACT

This disclosure describes systems for electrochemically sensing chemical species using nucleic acid aptamers and methods for using and fabricating these systems. This disclosure also describes a plurality of electrochemical sensors capable of measuring multiple chemical species.

### TECHNICAL FIELD

This invention relates to electrochemical sensors and methods of fabricating and using electrochemical sensors.

### BACKGROUND

Electrochemical sensors generally respond to chemical changes in a fluid, particularly to electrical potential, current, and capacitance at the sensor-fluid interface, i.e., the so-called electrochemistry. An electrical response in an electrochemical sensor results from these chemical changes. Typical electrochemical sensors, such as chemical-sensitive field-effect transistors (“ChemFETs”), can selectively detect some types of proteins in a fluid. Adsorption of a protein on a surface of the ChemFET causes a change in the electrical conductance of the ChemFET's electrical channel; this change can be related to the presence of the adsorbed protein.

To adsorb proteins, ChemFETs typically are coated with a protein antibody. Protein antibodies naturally have an affinity to and thus can adsorb particular proteins. There are significant problems with using protein antibodies with ChemFETs, however.

First, many important proteins cannot be adequately adsorbed by currently available protein antibodies. While some known protein antibodies are capable of adsorbing particular proteins, many important other proteins are not adsorbed or adsorbed with sufficient specificity to be effectively used with a ChemFET.

Second, protein antibodies are typically expensive to produce. One reason for this is that most protein antibodies are produced using animals. An animal naturally, or by human alteration produces a desired protein antibody. The protein antibody is then extracted from the animal and used with a ChemFET. As mentioned, this can be prohibitively expensive.

Third, protein antibodies are often prohibitively large for highly effective use in ChemFETs. Protein antibodies are generally on the order of ten or more nanometers in size. ChemFETs and other electrochemical sensors that use surface electrochemistry are more accurate and/or sensitive the closer the adsorbed protein or other chemical species is to the electrically active structure (e.g., a ChemFET's electrical channel). Because protein antibodies adsorb proteins at a significant distance from a ChemFETs electrical channel in part because of the protein antibodies' size, the ChemFET can have difficulty in accurately and/or sensitively measuring the presence of the adsorbed protein.

Further, the large size and low charge density of many protein targets exacerbate this problem. The size of an adsorbed protein acts to distance much of a protein's charge from an electrochemical sensor's electrically active structure. This distance can hinder sensitive measurement of the protein. Also, a protein's low charge density makes close proximity of the adsorbed protein to the electrically active surface especially important, but this close proximity is hindered by the size of a typical protein antibody.

Fourth, different protein antibody species may require different surface attachment chemistries. Fabricating a sensor that uses two or more different protein antibody species may be prohibitively expensive because of the additional multiplicative process cost and complexity of different surface attachment chemistries.

In addition, sensitive measurement of protein targets can be especially important. In many cases, bodily fluids having low protein concentrations need to be analyzed. Without sensitive measurement techniques, these low concentrations may not be measurable.

### BRIEF DESCRIPTION OF THE DRAWINGS

FIG. 1 illustrates a top plan view of an exemplary assembly of electrochemical sensors.

FIG. 2 illustrates an expanded clipped-plane view of the sensors of FIG. 1 and two expanded views of this clipped-plane view.

FIG. 3 illustrates the expanded views of FIG. 2 with an exemplary aptamer functionalizing layer formed there-over.

The same numbers are used throughout the disclosure and figures to reference like components and features.

### DETAILED DESCRIPTION

This document describes systems for electrochemically sensing chemical species using nucleic acid aptamers and methods for using and fabricating these systems. Various embodiments of electrochemical sensors usable in these systems and methods for fabricating them are described first below. Following this discussion, various embodiments of nucleic acid aptamers usable in these systems and methods for fabricating these aptamers are described.

Electrochemical Sensors

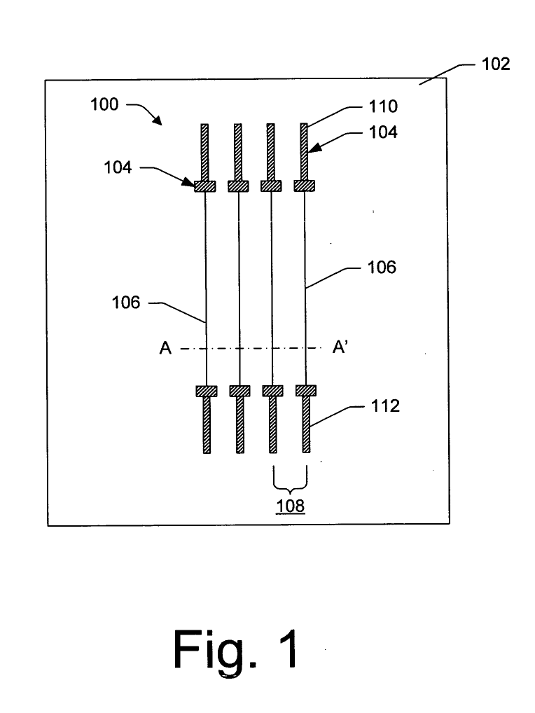

Referring to FIG. 1, an assembly of exemplary electrochemical sensors, shown generally at 100, are formed over a substrate 102. The assembly 100 comprises exemplary electrochemical sensors 104 having electrically active structures 106. The assembly 100 can be formed using photolithography, e-beam, or other suitable technique(s). In one embodiment, the assembly, sensors, and structures are formed using nano-imprint lithography. Nano-imprint lithography permits fabrication of features, such as the electrically active structures 106, having very small dimensions. In at least one embodiment, the dimensions (e.g., width or thickness) of the features are as small as about ten nanometers.

The assembly 100 can comprise a particular structure, such as an array or periodic array. In the ongoing embodiment, the assembly comprises a periodic array of the structures 106 having a pitch 108. The pitch is a distance between centers of elongate dimensions of the structures. Here the structures' pitch is less than or about ninety nanometers.

As formed, the electrically active structures 106 are capable of sensing an electric field at or near the structures. A conductance or impedance of the structures, for instance, can be altered by an electric field. This electric field can be caused by adsorption of chemical species in proximity to the structures.

In the ongoing embodiment, the electrochemical sensors 104 comprise chemical field-effect transistors (ChemFETs). In this embodiment, the electrically active structures 106 comprise electrical channels. These electrical channels can comprise semiconductive materials, such as lightly doped silicon, for instance.

The structures 106 are formed or positioned in electrical communication with source regions 110 and drain regions 112. These regions 110 and 112 can be formed in electrical communication with various devices capable of measuring, calibrating, and interacting with the sensors 104 (not shown).

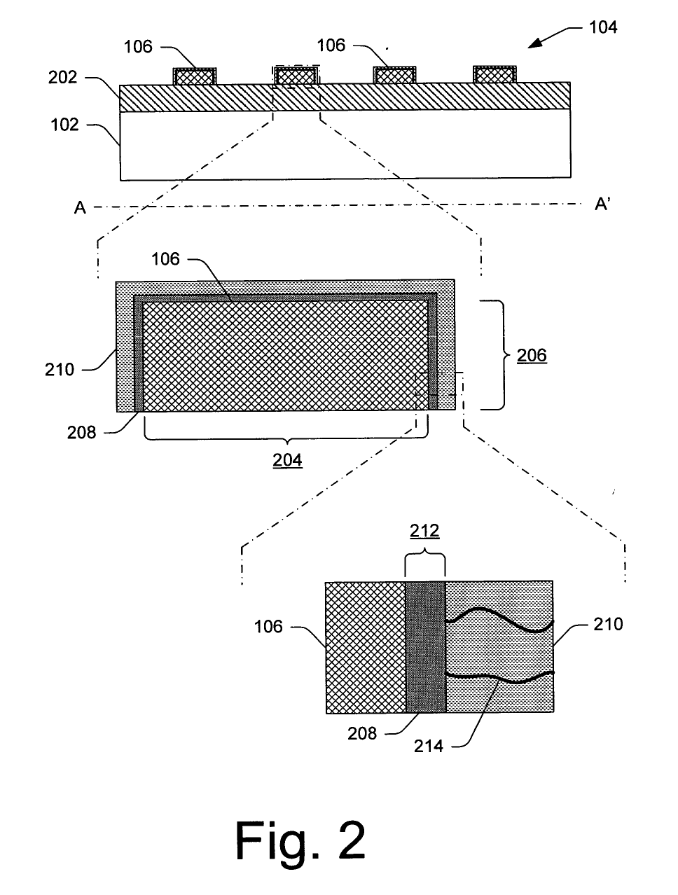

Referring to FIG. 2, an enlarged clipped-plane view along a line from A to A′ of FIG. 1 is shown. In the illustrated embodiment, substrate 102 supports the sensors 104 and can comprise, among other types of substrates, a semiconductive substrate. In the context of this document, the term “semiconductive substrate” is defined as any construction comprising semiconductive material, including, but not limited to, bulk semiconductive materials such as semiconductive or SOI wafer or chip and/or semiconductive material layers (both either alone or in assemblies comprising other materials). The term “substrate” refers to any supporting structure, including but not limited to, the semiconductive substrates described above. The illustrated and described substrate 102 comprises lightly doped silicon of about 10¹⁵ cm⁻³. An insulative layer 202 can be formed over the substrate 102 and can comprise a dielectric material, such as silicon dioxide. In the illustrated embodiment, the insulative layer 202 is about 200 nanometers thick.

An expanded clipped plane of one of the structures 106 is also shown. This view sets forth a section across the elongate dimension of the structures 106 (going into and out of the page). The cross-section shows the structure's width 204 and thickness 206. The width and/or the thickness can be from about ten nanometers to about ten microns. In the ongoing embodiment, the width is about forty nanometers and the thickness is about fifteen nanometers.

This expanded clipped plane shows a protective layer 208 formed over the structures 106. The protective layer can be electrically insulating and formed by oxidizing or nitriding the surface of the structures 106 or with another suitable technique. The protective layer is effective to protect the structure 106 from chemical attack by samples and their components that are to be analyzed, such as bodily fluids and chemical components present in bodily fluids. The protective layer can also act to improve adhesion or chemical bonding of a coupling layer 210, discussed below. The protective layer can additionally act to establish suitable interfacial bonding with the underlying semiconductor material so that minimal residual charge results at the interface. In the ongoing embodiment, the protective layer comprises a thin oxide, such as silicon dioxide or silicon nitride and is formed to a depth 212 (shown at another, further-expanded view) of about one nanometer. The depth of this layer 208 and other layers over the structure 106 can act to separate the structure from adsorbed chemical species, there by reducing the sensor's 104 sensitivity. Because of this, the layer 208 and other layers can be formed to thinly (i.e., with a small depth) cover the structure. In another embodiment, the protective layer comprises multiple layers, including at least one electrically insulating layer such as a thin oxide plus an additional layer of metal or nitride. The additional layer or layers provide additional or enhanced functionality, such as a nitride layer providing improved protection from chemical attack. A metal layer can also provide protection from chemical attack while not contributing to the insulating dielectric property of the total protective layer. This is because the metal transmits an electrical potential with negligible loss of the electric field strength. By so doing, the sensor can operate with a higher field strength.

The coupling layer 210 can be formed over the structures 106 through physical vapor deposition or another suitable technique. The coupling layer is effective to couple a nucleic acid aptamer (e.g., a deoxyribonucleic acid (DNA) or ribonucleic acid (RNA) or peptide nucleic acid (PNA) aptamer) to or over the structure. The coupling layer can aid in this coupling, though in some cases the aptamer can be coupled directly to the protective layer 208.

In the ongoing embodiment, the coupling layer 210 comprises (glycidoxypropyl)trimethoxy silane. This compound acts to couple or “tether” nucleic acid aptamers to or over the structure 106. This compound is shown as a tether 214 within the coupling layer 210. This compound, on one end, binds to the protective layer 208. On the other end, the compound has a strained ring which is chemically altered to yield a diol structure. This diol structure can then be reacted with carbonyldiimidazole. This carbonyldiimidazole provides an imidizole group on the outer end of the tether 214. This imidizole group can be reacted at this outer end with an amine-terminated nucleic acid aptamer. In this embodiment, the length of the tether 214 (i.e., the depth of the coupling layer 210) is about one to three nanometers, though it can, in some embodiments, be extended to about ten or more nanometers. This length/depth is small in dimension in order to permit the aptamer to be immobilized close to the structure 106 to permit greater sensitivity of the sensor 104.

Aptamers

Aptamers are oligonucleotides that exhibit molecular recognition capability. They can be engineered capable of adsorbing, binding to, or otherwise immobilizing chemical species such as peptides, proteins, epitopes on proteins, other nucleic acids, caffeine, organic dyes, and a variety of other molecular species.

Aptamers can be formed by various techniques which will be appreciated by the skilled artisan. One such technique is known as Systematic Evolution of Ligands by EXponential enrichment or “SELEX”. Aptamers fabricated using SELEX can be produced without use of animals, at relatively low cost, and in relatively large quantities.

Using a suitable technique, aptamers can be found that exhibit molecular recognition for many chemical species that are desired to be sensed using the sensor 104. In the ongoing embodiment, chemical species that are to be measured by the assembly 100 of the sensors 104 are immobilized to a surface. Using appropriate nucleic acid building blocks (e.g., A, G, T, C, and U), millions, billions, or trillions of nucleic acid aptamers are randomly generated, resulting in a combinatorial library of aptamers. These aptamers can be fabricated of various lengths having from a few bases to hundreds. In the ongoing embodiment, about ten to fifty bases are used, which translates to a length from about one to five nanometers. These aptamers are then exposed to the chemical species immobilized on the surface. Those that bind to the immobilized chemical species are retained and amplified. They can be amplified by various suitable techniques, including Polymerase Chain Reaction (PCR). With a large enough library of aptamers, a vast number of chemical species including small molecules, nucleic acids, peptides, proteins, and other biological macromolecules, can be measured using appropriate aptamers found using this technique.

An aptamer that interacts with an immobilized chemical species is generally capable of being used toward measuring that chemical species but also may be useful towards measurements of other chemical species. In some cases, two or more aptamers may exhibit selectivity towards one immobilized chemical species and thus each are capable of measuring that chemical species. In other cases, an aptamer may interact favorably with multiple chemical species. This aptamer alone is thus capable of being used in measuring any or all of these desired chemical species to which it demonstrates selectivity. Various examples of chemical species and their corresponding aptamers (RNA or ssDNA, PNAs not shown, exact base composition not shown) are set forth in Table 1 below.

TABLE 1

| Target Chemical Species | Aptamer |
|---|---|
| T4 DNA polymerase | RNA |
| Organic Dyes | ssDNA |
| L-arginine | RNA |
| FAD, FMN, NAD, NMN | RNA |
| Riboflavin, NMN | RNA |
| Human IgE (protein) | RNA, ssDNA |
| HIV Type I Rev (Residues 34-50) | RNA |
| L-selectin (protein) | ssDNA |
| SelB (E. coli protein) | RNA |
| S-adenosyl methionine | RNA |
| Ras binding-domain of Raf-1 (residues 51-131) | RNA |
| S-adenosyl homocysteine | RNA |
| Human oncostatin M (glycoprotein) | RNA |
| Ff gene 5 protein | ssDNA |

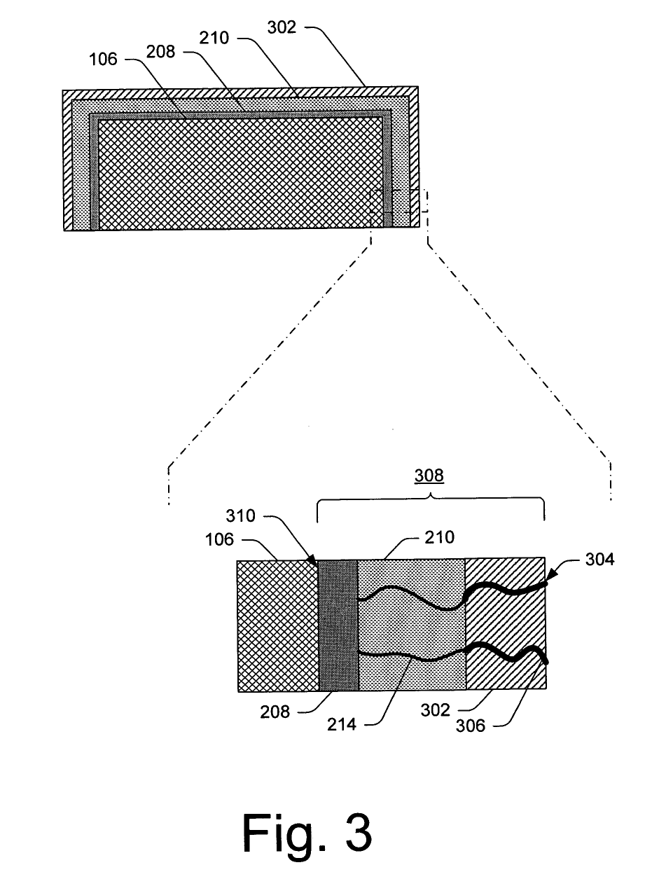

Referring to FIG. 3, an aptamer layer 302 is formed over the structure 106. The aptamer layer 302 functionalizes the sensor 104 by providing a chemically active surface 304. This active surface is formed capable of adsorbing, binding to, or otherwise immobilizing one or more chemical species to the aptamer layer 302. Viewed at a molecular level, this active surface comprises the binding regions of nucleic acid aptamer compounds 306 of the aptamer layer. The aptamer layer and the aptamer compounds can be coupled near the structure 106 by coupling to the coupling layer 210.

The aptamer layer 302 is shown here coupled to the coupling layer 210. In the ongoing embodiment, the nucleic acid aptamer compounds 306 are coupled to the imidizole groups on the outer ends of the tethers 214 (each tether representing a compound of the coupling layer 210). The aptamer layer 302, in this embodiment, is about one to about three nanometers in depth. Thus, each nucleic acid aptamer compound 306 of the aptamer layer 302 is about one to about three nanometers in length.

A distance 308 from the chemically active surface 304 of the aptamer layer 302 to the structure 106 is shown in FIG. 3. This distance 308 affects a sensitivity of the sensors 104 because the smaller the distance (when the surface 304 has immobilized a charged chemical species), the greater the electric field at the structure, and vice-versa. A very small distance enables high sensitivity of the structure and thus the sensor 104 to an electric field caused or affected by a chemical species immobilized on the aptamer layer 302.

In the ongoing embodiment, this distance 308 can be as little as about three to about nine nanometers including the protective layer 208 and the coupling layer 210. Use of an antibody of ten or more nanometers in size rather than a nucleic acid aptamer of about one nanometer in size would increase this distance to about twelve nanometers or more (if in both cases the protective layer 208 and the coupling layer 208 are about one nanometer in depth), or about four times the length. Because an electric field dissipates rapidly, this distance difference can reduce the strength of the electric field at a near edge 310 of the structure 106 by more than a factor of ten. This represents a significant difference in the electric field at the structure 106.

For an embodiment where each of the protective layer 208, the coupling layer 210, and the aptamer layer 302 are about three nanometers in depth, the distance 308 is about nine nanometers. For an antibody of about ten nanometers, the distance is an additional seven nanometers for a total of sixteen nanometers. In this embodiment, the strength of the electric field at the near edge 310 of the structure 106 is also a significant difference.

As is shown by these examples, the strength of an electric field at the structure 106 can be increased significantly by providing a smaller distance between a chemical species being measured and the structure.

For proteins, this difference in distance can be especially important. First, because proteins are large they generate a weaker electric-field strength effect at the structure 106 than similar but smaller chemical species because much of their charge is distanced from the structure. They also generate a weaker electric-field at the structure because the capacitive element of the electric field is reduced because of their large size (capacitance is inversely proportional to distance). Thus, this distance is especially important because the size of many proteins makes them more difficult to sense. Second, because some proteins of interest have low charge densities, measuring their presence is relatively difficult, further making a stronger electric field permitted by a small distance especially important. Third, because many solutions measured for proteins have very low concentrations of the proteins, sensing them can be relatively difficult because fewer charge carriers creates a weaker electric field. For these reasons, this reduced distance enabled by use of aptamers can be especially important when measuring proteins.

Some additional, exemplary ways in which to use aptamers to measure desired chemical species are set forth in the section entitled “Aptamer-Functionalized Arrays” below.

Aptamer-Functionalized Arrays

Various different aptamer layers 302 can be formed over each of the structures 106. As mentioned above, each of these layers 302 can comprise one or more aptamers capable of immobilizing one chemical species or multiple species. By selecting aptamer layers 302 that sense chemical species differently, it is possible to improve the sensing breadth, accuracy, and/or sensitivity of the assembly 100 of sensors 104.

In one embodiment, for instance, one of the structures 106 of the assembly is functionalized with a nucleic acid aptamer capable of immobilizing a chemical species and an interfering species. A neighboring structure 106 is functionalized with another nucleic acid aptamer capable of immobilizing the interfering species. For illustrative purposes the chemical species is called “X” and the interfering species is called “Y”. By so functionalizing, a measurement of the structure that is functionalized to sense “X” can be calibrated to adjust for the interfering chemical species “Y”. Also by so doing, the neighboring structure sensing “Y” may also reduce a concentration of “Y” at the other structure by attracting the chemical species “Y”. This can reduce the interference of “Y” on the structure that is functionalized to sense “X”. In this embodiment, the measurement accuracy is improved, and the occurrence of false-positive errors is reduced.

In another embodiment, one of the structures 106 is functionalized with a nucleic acid aptamer capable of sensing a chemical species called, for illustrative purposes, “Z” and another of the structures 106 is functionalized also to sense the species “Z” but with a different functionalizing agent. By so doing, a more accurate account of the species “Z” can be obtained. Further, if either of the first and second functionalizing agent are sensitive to an interfering species (called “AA”), but the other is not, a more accurate accounting of the species “Z” can be found by calibrating for the interference of “AA”.

In yet another embodiment, three of the structures 106 are functionalized with different nucleic acid aptamers, each capable of immobilizing the same chemical species but in different ways. By so doing, a more accurate measure of the concentration of that chemical species is possible. Problems with any one of the aptamers, such as unknown or known interfering agents, can have less of a negative effect on the total reading of the assembly 100.

In still another embodiment, one of the structures 106 is functionalized with a nucleic acid aptamer capable of immobilizing all of the desired chemical species. The other structures 106 are functionalized with nucleic acid aptamers that measure one or more of the desired chemical species. Thus, if “X”, “Y”, and “Z” are desired to be measured, one of the structures can measure the total of “X”, “Y”, and “Z”, another just “X”, and another “Y” and “Z”. By so doing, a concentration of the total and of each of “X”, “Y”, and “Z” can be determined. By functionalizing the structures of the assembly 100 in this manner, these concentrations can be determined without having an aptamer that can sense just “Y” or just “Z”.

These aptamer-functionalized electrochemical sensors have many important applications. Assume, for instance, that three known proteins are indicators for breast cancer and can be present in a person's blood or other bodily fluid. Assume also that if cancer-indicator “A” is of the highest concentration of the three, that one particular chemotherapy drug works best to save that person's life. These aptamer-functionalized electrochemical sensors can be capable of determining if a person has breast-cancer indicators to a higher sensitivity and/or accuracy than many antibody-functionalized electrochemical sensors. Also, in some cases these aptamer-functionalized sensors may be able to sense a breast-cancer-indicating protein that can not currently be sensed at all by typical electrochemical sensors. Further, these aptamer-functionalized sensors can be capable of determining with a high degree of sensitivity various concentrations of those proteins (e.g., a high concentration of cancer-indicator “A”), which may improve a person's prognosis by enabling her doctor to prescribe a better chemotherapy drug. This is just one example of a way in which these aptamer-functionalized electrochemical sensors can be used to good effect.

Although the invention is described in language specific to structural features and methodological steps, it is to be understood that the invention defined in the appended claims is not necessarily limited to the specific features or steps described. Rather, the specific features and steps disclosed represent preferred forms of implementing the claimed invention.

What is claimed is:

1. An apparatus comprising:
a plurality of electrochemical sensors having electrically active structures, the electrically active structures each having a conductance or impedance capable of being altered by an electric field; and
functionalized layers in proximity with the electrically active structures and comprising nucleic acid aptamers, the nucleic acid aptamers capable of modifying an electric field by binding one or more chemical species,
wherein one of the functionalized layers comprises a chemically active surface at which one or more of the chemical species can be immobilized within less than or about nine nanometers from one of the electrically active structures.

2. The apparatus of claim 1, wherein the chemically active surface is about one to about three nanometers from one of the electrically active structures.

3. The apparatus of claim 1, wherein one or more of the functionalized layers is about one to about three nanometers in depth.

4. The apparatus of claim 1, wherein a first of the functionalized layers comprises a first aptamer capable of immobilizing a first chemical species and a second of the functionalized layers comprises a second aptamer capable of immobilizing a second species.

5. The apparatus of claim 4, wherein the first aptamer is further capable of immobilizing the second species.

6. The apparatus of claim 4, wherein a third of the functionalized layers comprises a third aptamer capable of immobilizing the first species, the second species, or a third species.

7. The apparatus of claim 1, wherein a first of the functionalized layers comprises a first aptamer capable of immobilizing a species and a second of the functionalized layers comprises a second aptamer capable of immobilizing the species.

8. The apparatus of claim 1, wherein two or more of the functionalized layers comprise identical nucleic acid aptamers.

9. The apparatus of claim 1, wherein the plurality comprises a periodic array.

10. The apparatus of claim 9, wherein the plurality has a pitch of less than or about ninety nanometers.

11. The apparatus of claim 1, wherein one of the electrochemical sensors comprises a chemical field-effect transistor (ChemFET).

12. The apparatus of claim 1, wherein one of the electrically active structures has a width or thickness from about ten to about ninety nanometers.

13. The apparatus of claim 1, further comprising a protective layer over one or more of the electrically active structures, the protective layer capable of protecting the electrically active structure from chemical attack by a sample and its components.

14. The apparatus of claim 1, further comprising a coupling layer over one or more of the electrically active structures, the coupling layer capable of coupling the nucleic acid aptamers in proximity to the electrically active structure.

15. The apparatus of claim 1, wherein the charged chemical species comprises one or more proteins, peptides, or other biological macromolecules.

16. An electrochemical sensor comprising:
an electrically active structure in electrical communication with source and drain regions, the electrically active structure having a conductance capable of being altered by an electric field; and
an aptamer layer of about one to three nanometers in depth that is in proximity with the electrically active structure and comprises a nucleic acid aptamer, the nucleic acid aptamer capable of creating an electric field by immobilizing a charged chemical species.

17. The sensor of claim 16, wherein the sensor comprises a chemical field-effect transistor (ChemFET).

18. The sensor of claim 16, wherein the electrically active structure has a dimension from about ten to about ninety nanometers.

19. The sensor of claim 16, further comprising a protective layer over the electrically active structure, the protective layer capable of protecting the electrically active structure from chemical attack by a sample and its components.

20. The sensor of claim 19, wherein the protective layer is about one to about three nanometers in depth.

21. The sensor of claim 16, further comprising a coupling layer over the electrically active structure, the coupling layer capable of coupling the nucleic acid aptamer in proximity to the electrically active structure.

22. The sensor of claim 16, wherein the charged chemical species comprises a protein, peptide or other biological macromolecule.

23. The sensor of claim 16, wherein the nucleic acid aptamer is ribonucleic acid.

24. The sensor of claim 16, wherein the nucleic acid aptamer is deoxyribonucleic acid.

25. The sensor of claim 16, wherein the aptamer is a peptide nucleic acid.

26. The sensor of claim 16, wherein the aptamer layer comprises a chemically active surface at which the charged chemical species can be immobilized and a distance between the chemically active surface and the electrically active structure is less than or about nine nanometers.

27. The sensor of claim 26, wherein the distance is less than or about three nanometers.

28. A method comprising:
providing an electrically active structure having a conductance or impedance capable of being altered by an electric field; and
immobilizing within about six nanometers of the electrically active structure a nucleic acid aptamer that is capable of immobilizing a chemical species.

29. The method of claim 28, wherein the chemical species comprises a protein, peptide or other biological macromolecule.

30. The method of claim 28, wherein the nucleic acid aptamer is about ten to about fifty nucleic-acid bases.

31. The method of claim 28, wherein the nucleic acid aptamer is about one to about three nanometers in length.

32. The method of claim 28, further comprising:
providing additional electrically active structures; and
immobilizing within about six nanometers of the additional electrically active structures additional nucleic acid aptamers capable of immobilizing additional chemical species.

33. A method comprising:
forming an electrically active structure of an electrochemical sensor over a substrate;
forming a protective layer over the electrically active structure effective to protect the electrically active structure from chemical attack by a fluid desired to be analyzed;
forming a coupling layer over the electrically active structure capable of coupling to a nucleic acid aptamer; and
coupling a layer comprising nucleic acid aptamers to the coupling layer, the aptamer layer capable of immobilizing a chemical species within about nine nanometers of the protective layer.

34. The method of claim 33, wherein the act of forming the electrically active structure comprises nano-imprint lithography.

35. The method of claim 34, wherein the act of forming the electrically active structure comprises forming the electrically active structure with a width of about ten to about ninety nanometers.

36. The method of claim 33, wherein the substrate over which the electrically active structure is formed comprises a silicon-on-insulator (SOI) wafer.

37. The method of claim 33, wherein the protective layer is formed having a depth of about one to about three nanometers.

38. The method of claim 33, wherein the protective layer is formed comprising an electrically insulating layer and an electrically conducting layer.

39. The method of claim 33, wherein the coupling layer is formed having a depth of about one to about three nanometers.

40. The method of claim 33, wherein the aptamer layer is formed having a depth of about one to about three nanometers.

41. The method of claim 33, further comprising forming nucleic acid aptamers of the aptamer layer using Systematic Evolution of Ligands by EXponential enrichment (SELEX).

42. The method of claim 33, wherein the chemical species comprises a protein, peptide or other biological macromolecule.

43. A ChemFET comprising:
an electrical channel having a conductance capable of being altered by an electric field; and
a functionalized layer having a chemically active surface, the chemically active surface capable of immobilizing a small molecule, protein, peptide or other biological macromolecule and positioned within about nine nanometers of the electrical channel or an electrically conductive structure in electrical communication with the electrical channel.

44. The ChemFET of claim 43, wherein the electrical channel has a dimension from about ten to about ninety nanometers.

45. The ChemFET of claim 43, further comprising a protective layer positioned between the electrical channel and the functionalized layer and capable of protecting the electrical channel from chemical attack by a sample and its components.

46. The ChemFET of claim 45, wherein the protective layer comprises the electrically conductive structure in electrical communication with the electrical channel.

47. The ChemFET of claim 43, further comprising a coupling layer positioned between the electrical channel and the functionalized layer and capable of coupling the functionalized layer over the electrical channel.

48. The ChemFET of claim 43, wherein the functionalized layer comprises one or more nucleic acid aptamers.

49. The ChemFET of claim 43, wherein the chemically active surface is positioned within about three nanometers of the electrical channel.

50. The ChemFET of claim 43, wherein the ChemFET is oriented in an array comprising a plurality of other ChemFETs.

51. The ChemFET of claim 50, wherein one of the other ChemFETs of the array is capable of immobilizing the small molecule, protein, peptide or other biological macromolecule.

52. The ChemFET of claim 50, wherein one of the other ChemFETs of the array is capable of immobilizing a different small molecule, protein, peptide or other biological macromolecule.

53. The ChemFET of claim 50, wherein one of the other ChemFETs of the array is capable of immobilizing a different chemical species than the small molecule, protein, peptide or other biological macromolecule, and that is not a protein.
